(19) United States
(12) Patent Application Publication
(10) Pub. No.: US 2023/0364049 A1
(43) Pub. Date: Nov. 16, 2023

(54) COMBINATION OF P2Y6 INHIBITORS AND IMMUNE CHECKPOINT INHIBITORS

(71) Applicants: VIB VZW, Gent (BE); KATHOLIEKE UNIVERSITEIT LEUVEN, K.U.LEUVEN R&D, Leuven (BE)

(72) Inventor: Massimiliano MAZZONE, Kessel-Lo (BE)

(21) Appl. No.: 18/246,585

(22) PCT Filed: Sep. 24, 2021
(86) PCT No.: PCT/EP2021/076296

§ 371 Date: Mar. 24, 2023

(30) Foreign Application Priority Data
Sep. 24, 2020 (GB) 2015082.7
Jan. 29, 2021 (EP) 21154226.1

(51) Int. Cl. A61K 31/26; A61K 39/395; A61K 39/00; A61P 35/00

(52) U.S. Cl. CPC A61K 31/26; A61K 39/3955; A61K 39/4646; A61K 39/4614; A61P 35/00

(57) ABSTRACT

The invention is situated in the field of cancer treatment. In particular it relates to treatments comprising combining an inhibitor of the pyrimidinergic receptor P2Y6 and an immune checkpoint inhibitor. Further in particular, the treatment is of benefit for cancers poorly responding to immune checkpoint inhibitor therapy.

### CROSS-REFERENCE TO RELATED APPLICATIONS

This application is a national phase entry under 35 U.S.C. § 371 of International Patent Application PCT/EP2021/076296, filed Sep. 24, 2021, designating the United States of America and published in English as International Patent Publication WO 2022/063947 on Mar. 31, 2022, which claims the benefit under Article 8 of the Patent Cooperation Treaty to United Kingdom Patent Application Serial No. 2015082.7, filed Sep. 24, 2020, and to European Patent Application Serial No. 21154226.1, filed Jan. 29, 2021, the entireties of which are hereby incorporated by reference.

### FIELD OF THE INVENTION

The invention is situated in the field of cancer treatment. In particular it relates to treatments comprising combining an inhibitor of the pyrimidinergic receptor P2Y6 and an immune checkpoint inhibitor. Further in particular, the treatment is of benefit for cancers poorly responding to immune checkpoint inhibitor therapy.

### BACKGROUND OF THE INVENTION

Cancer immunotherapy has provided patients with a promising treatment option. Therapeutic regimens such as adoptive T cell transfer (ACT), cancer vaccines and immune checkpoint inhibitors (e.g. anti-PD-1 or anti-CTLA-4 antibodies), harness the ability of the immune system to recognize and reject the tumor (Smyth et al. 2015, Nat Rev Clin Oncol 13:143-158). However, despite high response rates, with prolonged survival in a subset of melanoma (e.g. Schadendorf et al. 2015, J Clin Oncol 33:1889-189), lung (e.g. Borghaei et al. 2015, N Engl J Med 373:1627-1639), and renal cancer patients (e.g. Motzer et al. 2015, N Engl J Med 373:1803-1813), for several other tumors such as mismatch repair (MMR)-proficient colorectal cancer (CRC) (e.g. Le et al. 2015, N Engl J Med 372: 1509-2520) and pancreatic ductal adenocarcinoma (PDAC) (e.g. Sarantis et al. 2020, World J Gastrointest Oncol 12: 173-181) immunotherapy fails to show any clinical benefit.

PDAC is one of the most aggressive and lethal cancer types. The projected doubling of the incidence of PDAC by 2030 would make it the second most common cause of cancer related death, following lung cancer. Tumors develop rapidly, invading surrounding tissues with the consequence that fewer than 20% of the patients are eligible for resection at the moment of diagnosis (Pereira et al. 2020, The Lancet Gastroentrol Hepatol 5:698-710). Most of the therapies including the recent immunotherapeutic approaches are not effective (Royal et al. 2010, J Immunother 33:828-833), and the majority of those patients that do proceed with surgery will ultimately relapse (Strobel et al. 2017, Ann Surg 265:565-573; Kamisawa et al. 2016, The Lancet 388:73-85). Therefore, there is an urgent need for treatments applicable to the vast majority of patients with unresectable tumors or that prevent post-surgical relapse (Neoptolemos et al. 2017, The Lancet 389:1011-1024). PDACs are characterized by a dense desmoplastic stroma that impedes oxygen and nutrient diffusion from the blood stream and contributes to a strong hypoxic and acidic tumor microenvironment (TME) (Gajewski et al. 2013, Nat Immunol 14:1014-1022; Whatcott et al. 2015, Clin Cancer Res 21:3561-3568). In this harsh TME, cytotoxic T cells struggle to enter or to work efficiently (Joyce et al. 2015, Science 348, 74-80), also because pancreatic cancer cells are poorly recognized by the immune system due to the downregulation of the major histocompatibility complex class I (Yamamoto et al. 2020, Nature 581:100-105). Preclinical and clinical efforts have been pursued to make pancreatic tumors more immunogenic. These efforts encompass the combination of immune checkpoint inhibitors with pharmacological strategies targeting immunosuppressive fibroblasts, myeloid cells, or regulatory T cells, as well as cancer vaccines (e.g. GVAX) genetically modified to release immune stimulatory cytokines (e.g. Jaffee et al. 2001, J Clin Oncol 19:145-156; Lutz et al. 2011, Ann Surg 253:328-335; Ozdemir et al. 2014, Cancer Cell 25:719-734; Rhim et al. 2014, Cancer Cell 25:735-747; Elyada et al. 2019, Cancer Discov 9:1102-1123; Mantovani et al. 2017, Nat Rev Clin Oncol 14:399-416; Huelsken & Hanahan 2018, Cell 172:643-644; Zhu et al. 2017, Immunity 47:323-338). Nevertheless, none of these approaches has reached the desired effects so far.

P2Y6 is a specific high affinity receptor for UDP and only reacts little to UTP and UMP. The role of the pyrimidinergic receptor P2Y6 (P2Y6R) in the immune response or in cancer has been described at the level of dendritic cells (DCs: Cammarata et al. 2016, J Med Dev Sci 2:30-37), macrophages (Bar et al. 2008, Mol Pharmacol 74: 777-784) and T-cells (Tsukimoto et al. 2009, BBRC 384: 512-518),In vivo tumor growth has been reported to be inhibited by P2Y6 activation (gastric tumor; Wan et al. 2017, Sci Rep 7: 2459; myeloid neoplasias: WO2015165975A1) or by P2Y6 inhibition (breast cancer metastasis; Ma et al. 2016, Oncotarget 7: 29036-29050; colorectal: Placet et al. 2018, Biochim Biophys Acta Mol Basis Dis 1864(5 Pt A):1539-1551; melanoma metastasis: Qin et al. 2020, Cell Mol Immunol doi:10.1038/s41423-020-0392-0; see also WO2017070660A1 and US20180271863A1). It was further reported that P2Y6 appears intrinsically linked to the neoplastic process (Stoll et al. 2018, Oncoimmunol 7: e1484980).

### SUMMARY OF THE INVENTION

The invention in one aspect relates to an inhibitor of the pyrimidinergic receptor P2Y6 (P2Y6) for use in treating or inhibiting cancer, or for use in inhibiting progression of cancer, wherein the treatment or inhibition is in combination with an immune checkpoint inhibitor.

In a further aspect, the invention relates to an immune checkpoint inhibitor for use in treating or inhibiting cancer, or for use in inhibiting progression of cancer, wherein the treatment or inhibition is in combination with an inhibitor of P2Y6.

In another aspect, the invention covers an inhibitor of P2Y6 and an immune checkpoint inhibitor for use in treating or inhibiting cancer, or for use in inhibiting progression of cancer.

The invention also relates to combinations of an inhibitor of the pyrimidinergic receptor P2Y6 (P2Y6) and an immune checkpoint inhibitor.

In a further aspect, the invention relates to isolated P2Y6 knock-out macrophages or to isolated macrophages conditionally expressing a P2Y6 inhibitor. These are e.g. for use such as for use as a medicament, ore for use in treating or inhibiting cancer, or for use in inhibiting progression of cancer. These may be included in a pharmaceutical composition further comprising an excipient. These may also be included in a combination with an immune checkpoint inhibitor; and such combinations are e.g. for use such as for use as a medicament, ore for use in treating or inhibiting cancer, or for use in inhibiting progression of cancer.

In any of the above, the cancer is in particular a cancer that is not or only partially responding to immune checkpoint inhibitor therapy.

In any of the above, the inhibitor of P2Y6 may be a specific inhibitor of P2Y6, such as a genetic or pharmacological inhibitor of P2Y6.

In any of the above, the immune checkpoint inhibitor may in particular be an inhibitor of PD-1.

### FIGURE LEGENDS

FIGS. 1A and 1B. Relative expression of different P2-type receptors and of the housekeeping gene Hypoxanthine Phosphoribosyltransferase (Hprt) as indicated on the X-axis. Expression was determined in murine bone marrow derived macrophages (BMDMs), in murine peritoneal exudate macrophages (PEMs), in tumor-associated macrophages (TAMs) from mice harboring 4T1 mammary carcinoma (4T1) or Lewis lung carcinoma (LLC), and in human breast cancer cells.

FIG. 2. Relative expression of P2ry6 based on single-cell RNA sequencing (scRNAseq) in the cell populations indicated on the X-axis. Cell populations were isolated from mice harboring the KPC pancreatic tumor.

FIG. 3. FACS quantification (MFI: mean fluorescence intensities) of P2Y6 expression in the tumor compartment of tumor-bearing mice.

FIG. 4. Migration of murine bone marrow derived macrophages (BMDMs). Migration of BMDMs is enhanced in the presence of UDP as chemoattractant. The enhanced migration of BMDMs towards UDP is abrogated by the selective P2Y6 inhibitor MRS2578.

FIG. 5. Reduction in Panc02 tumor volume (left panel) and tumor weight (right panel) upon treatment with control antibody (IgG), anti-PD1 antibody (α-PD-1), the selective P2Y6 inhibitor MRS2578, and the combination of the selective P2Y6 inhibitor MRS2578 with the anti-PD1 antibody. Timing of administration of the anti-PD1 antibody is indicated by black arrows.

FIG. 6. FACS quantification of M2 macrophages (via CD206, in F4/80+ cells), granzyme B (GZMB) in CD8+ T cells, and of IFNγ in CD8+ T cells. Cells were isolated from mice harboring untreated Panc02 tumors (vehicle) and from mice harboring Panc02 tumors treated with the selective P2Y6 inhibitor MRS2578 (MRS2578).

FIG. 7. Evolution of body weight of mice treated as described for FIG. 5.

FIGS. 8A and 8B. (FIG. 8A) Migration of murine bone marrow derived macrophages (BMDMs). Migration of BMDMs is enhanced in the presence of UDP as chemoattractant. The enhanced migration of BMDMs towards UDP is abrogated by genetic inhibition of P2Y6, but not by genetic inhibition of P2Y14. (FIG. 8B) Genetic inhibition of P2Y6 and P2Y14 as achieved in BMDMs by means of siRNA.

FIGS. 9A-9C. (FIG. 9A) Growth of KPC tumor in mice after adoptive transfer of wild-type macrophages (“WT”) or of macrophages in which P2Y6 is subsequently conditionally knocked out “KO” in combination with administration of a control antibody (“IgG”) or of anti-PD-1 antibody (“anti PD-1”). Macrophages were adoptively transferred by means of bone marrow transplantation. (FIG. 9B) Mesenteric metastasis in mice of (FIG. 9A). (FIG. 9C) Polarization of tumor-associated macrophages (TAMs) in mice of (FIG. 9A).

### DETAILED DESCRIPTION

In work leading to the current invention, pyrimidinergic receptor P2Y6 (or P2YR6) was identified as a potential target involved in tumor resistance to immune checkpoint inhibitors as P2Y6 blockade (pharmacological and genetic blockade; genetic blockade in particular in macrophages) promoted the response to anti-PD-1 in pancreatic ductal adenocarcinoma (PDAC). This P2Y6 receptor therefore constitutes a pharmacological target in cancer immunotherapy.

Therefore, the invention in one aspect relates to an inhibitor of the pyrimidinergic receptor P2Y6 for use in treating or inhibiting cancer or for use in inhibiting progression of cancer, in combination with (administration of) an immune checkpoint inhibitor, or wherein the treatment or inhibition (by therapy including a P2Y6 inhibitor) is combined with immune checkpoint inhibitor therapy or with administration of an immune checkpoint inhibitor.

Alternatively, the invention relates to use of an inhibitor of P2Y6 in the manufacture of a medicament for use in combination with an immune checkpoint inhibitor for treating or inhibiting cancer or for inhibiting progression of cancer (in a subject or individual having the cancer). Alternatively, the invention relates to use of an inhibitor of P2Y6 in the manufacture of a medicament for treating or inhibiting cancer or for inhibiting progression of cancer (in a subject or individual having the cancer) in combination with an immune checkpoint inhibitor (for treating or inhibiting cancer or for inhibiting progression of cancer), or in combination with administering an immune checkpoint inhibitor to the subject or individual, or wherein the treatment or inhibition (by therapy including a P2Y6 inhibitor) is combined with immune checkpoint inhibitor therapy.

In an alternative aspect, the invention relates to an immune checkpoint inhibitor for use in treating or inhibiting cancer or for use in inhibiting progression of cancer, in combination with (administration of) an inhibitor of P2Y6, or wherein the treatment or inhibition (by therapy including an immune checkpoint inhibitor) is combined with P2Y6 inhibitor therapy or with administration of an inhibitor of P2Y6. Alternatively, the invention relates to use of an immune checkpoint inhibitor in the manufacture of a medicament for use in combination with an inhibitor of P2Y6 for treating or inhibiting cancer or for inhibiting progression of cancer (in a subject or individual having the cancer). Alternatively, the invention relates to use of an immune checkpoint inhibitor in the manufacture of a medicament for treating or inhibiting cancer or for inhibiting progression of cancer (in a subject or individual having the cancer) in combination with an inhibitor of P2Y6 (for treating or inhibiting cancer or for inhibiting progression of cancer), or in combination with administering an P2Y6 inhibitor to the subject or individual, or wherein the treatment or inhibition (by therapy including an immune checkpoint inhibitor) is combined with P2Y6 inhibitor therapy.

In another alternative aspect, the invention relates to an inhibitor of P2Y6 and an immune checkpoint inhibitor for use in treating or inhibiting cancer or for use in inhibiting progression of cancer. Alternatively, the invention relates to use of an inhibitor of P2Y6 and (use of) an immune checkpoint inhibitor in the manufacture of a medicament for use in treating or inhibiting cancer or for inhibiting progression of cancer (in a subject or individual having the cancer).

A further aspect of the invention relates to methods for treating or inhibiting cancer, or a method for inhibiting progression of cancer, in a subject or individual (in particular a mammalian subject or mammal, such as a human subject or human), the methods comprising administering an inhibitor of P2Y6 and administering an immune checkpoint inhibitor to the subject or individual. By administering the inhibitor of P2Y6 and the immune checkpoint inhibitor, the cancer is treated or inhibited, or the progression of the cancer is inhibited. In particular, an effective amount of the inhibitor of P2Y6 and of the immune checkpoint inhibitor is administered to the subject; or an effective amount of a combination (in any way) of the inhibitor of P2Y6 and of the immune checkpoint inhibitor is administered to the subject.

In any of the above aspects and embodiments, the combination is in particular a combination in any way or in any appropriate way (explained in more detail hereinafter).

In the above, the inhibitor of P2Y6 may in particular be a specific inhibitor of P2Y6 (see further in explanation of genetic/pharmacological inhibitors).

In any of the above aspects and embodiments, the inhibitor of P2Y6 may be a genetic inhibitor of P2Y6 or a pharmacological inhibitor of P2Y6.

In particular, a genetic inhibitor of P2Y6 is an inhibitor interfering with P2Y6 gene expression. A genetic inhibitor of P2Y6 may be a DNA nuclease specifically knocking out or disrupting P2Y6/the P2Y6 gene, an RNase specifically targeting P2Y6/P2Y6 transcripts, or an inhibitory oligonucleotide specifically targeting P2Y6/P2Y6 transcripts. Such DNA nuclease specifically knocking out or disrupting P2Y6/the P2Y6 gene may be selected from (the group consisting of) a ZFN, a TALEN, a CRISPR-Cas, and a meganuclease. Such RNase specifically targeting P2Y6/P2Y6 transcripts may be selected from (the group consisting of) a ribozyme and a CRISPR-C2c2. Such inhibitory oligonucleotide specifically targeting P2Y6/P2Y6 transcripts may be selected from (the group consisting of) an antisense oligomer, a siRNA, a shRNA, and gapmer. In particular, a pharmacological inhibitor of P2Y6 may be selected from (the group consisting of) a polypeptide comprising an immunoglobulin variable domain, a monoclonal antibody or a fragment thereof, an alpha-body, a nanobody, an intrabody, an aptamer, a DARPin, an affibody, an affitin, an anticalin, a monobody, a bicyclic peptide, a PROTAC, or a LYTAC. The pharmacological inhibitor of P2Y6 may also be selected from small molecule inhibitors.

In any of the above aspects and embodiments, the immune checkpoint inhibitor is an inhibitor of (the group consisting of) PD1, PDL1, PDL2, CTLA4, B7-1, B7-2, A2AR, B7-H3 (or CD276), B7-H4 (or VTCN1), BTLA (or CD272), IDO, KIR, LAG3, NOX2, TIM3, VISTA, SIGLEC7 (or CD328), or SIGLEC9 (see further).

In any of the above aspects and embodiments, the cancer or tumor in particular is a cancer or tumor that is poorly responding to or resistant to immune checkpoint inhibitor therapy. Poor response or resistance to immune checkpoint inhibitor therapy is herewith understood as either non-response (NR) or partial response (PR) to immune checkpoint inhibitor therapy, in particular to a therapy consisting of administration of immune checkpoint inhibitor only, or in particular to a therapy comprising administration of immune checkpoint inhibitor but not comprising or excluding administration of a P2Y6 inhibitor. The poor response, resistance, non-response or partial response in particular may be based on clinical experience. In particular, the cancer can be pancreatic cancer. The therapy comprising an immune checkpoint inhibitor may in particular be a therapy comprising a single immune checkpoint inhibitor.

P2Y6 and P2Y6 Inhibitors

Aliases of P2Y6 provided in GeneCards® include P2RY6; P2Y6 receptor; pyrimidinergic receptor P2Y6; pyrimidinergic Receptor P2Y, G-Protein Coupled, 6; P2Y Purinoceptor 6; G-Coupled Nucleotide Receptor; P2 Purinoceptor. The genomic locations for the P2Y6 gene are chr11:73,264,503-73,298,625 (in GRCh38/hg38) and chr11:72,975,550-73,009,664 (in GRCh37/hg19). The GenBank reference P2Y6 mRNA sequences are known under accession nos. NM_001277204.2; NM_001277205.1; NM_001277206.1; NM_001277207.1; NM_001277208.1; NM_004154.3; NM_176796.2; NM_176797.2; and NM_176798.2. Human P2Y6 shRNA and human P2Y6 shRNA lentiviral particles are offered for sale by e.g. Origene. Pharmacological inhibitors of P2Y6 include selective small molecule inhibitors or antagonists such as TIM-38 (3-nitro-2-(trifluoromethyl)-2Hchromene, PubChem CID: 16762479), and variants of TIM-38: Compound 1 (6-bromo-3-nitro-2-(trifluoromethyl)-2H-chromene, PubChem CID: 11681366); Compound 2 (2-(4-chlorophenyl)-3-nitro-2Hchromene, PubChem CID: 3774501); Compound 3 (2-(4-methoxyphenyl)-3-nitro-2H-chromene, PubChem CID: 2732134); and Compound 4 (3-nitro-2-phenyl-2H-chromene, PubChem CID: 42450) (Ito et al. 2017, Life Sci 180:137-142). Other selective P2Y6 inhibitors or antagonists include the non-nucleotide di-isothiocyanate derivative N,N-1,4-butanediylbis-N-(3-isothiocyanatophenyl) thiourea (MRS2578), MRS2567, and MRS2575 (Mamedova et al. 2004, Biochem Pharmacol 67:1763-1770). Other genetic and pharmacological inhibitors of P2Y6 are described hereinafter.

Immune Checkpoint Inhibitors

Immunotherapy is a promising new area of cancer therapeutics and several immunotherapies have been approved or are being evaluated preclinically as well as in clinical trials and have demonstrated promising activity (Callahan et al. 2013, J Leukoc Biol 94:41-53; Page et al. 2014, Annu Rev Med 65:185-202). Immunotherapeutic agents include immune checkpoints antagonists including the cell surface protein cytotoxic T lymphocyte antigen-4 (CTLA-4) and programmed death-1 (PD-1) with their respective ligands. CTLA-4 binds to its co-receptor B7-1 (CD80) or B7-2 (CD86); PD-1 binds to its ligands PD-L1 (B7-H10) and PD-L2 (B7-DC). Other immune checkpoint inhibitors include the adenosine A2A receptor (AZAR), B7-H3 (or CD276), B7-H4 (or VTCN1), BTLA (or CD272), IDO (indoleamine 2,3-10 dioxygenase), KIR (killer-cell immunoglobulin-like receptor), LAG3 (lymphocyte activation gene-3), NOX2 (nicotinamide adenine dinucleotide phosphate (NADPH) oxidase isoform 2), TIM3 (T-cell immunoglobulin domain and mucin domain 3), VISTA (V-domain Ig suppressor of T cell activation), SIGLEC7 (sialic acid-binding immunoglobulin-type lectin 7, or CD328) and SIGLEC9 (sialic acid-binding immunoglobulin-type lectin 9, or CD329). Inhibition of immune checkpoints proteins can, in a subset of cancers, reactivate the subject's immune system towards cancer cells. Different inhibitors of PD1, PDL1 and CTLA4 have meanwhile received marketing approval, and detailed information on these 3 immune checkpoint proteins is included hereafter. Means of inhibition of immune checkpoint proteins are clearly not limited to inhibitors that received marketing approval.

In referring to genes or proteins herein (including to the P2Y6 gene or protein), no distinction is made in the annotation. Thus, whereas for example the human PD1 gene would be referred to as the PDCD1 gene, the mRNA as PDCD1 mRNA, and the protein as PDCD1, such distinction is not made hereinabove or hereinafter.

PD1

Aliases of PD1 provided in GeneCards® include PDCD1; Programmed Cell Death 1; Systemic Lupus Erythematosus Susceptibility 2; PD-1; CD279; HPD-1; SLEB2; and HPD-L. The genomic locations for the PDCD1 gene are chr2:241,849,881-241,858,908 (in GRCh38/hg38) and chr2:242,792,033-242,801,060 (in GRCh37/hg19). The GenBank reference PD1 mRNA sequence is known under accession no. NM_005018.3. Approved PD1-inhibiting antibodies include nivolumab, pembrolizumab, and cemiplimab; PD1-inhibiting antibodies under development include CT-011 (pidilizumab) and therapy with PD1-inhibiting antibodies is referred to herein as α-PD-1 therapy or α-PD1 therapy. PD1 siRNA and shRNA products are available through e.g. Origene.

PD-L1

Aliases of PD-L1 provided in GeneCards® include CD274, Programmed Cell Death 1 Ligand 1, B7 Homolog 1, B7H1, PDL1, PDCD1 Ligand 1, PDCD1LG1, PDCD1L1, HPD-L1, B7-H1, B7-H, and Programmed Death Ligand 1. The genomic locations for the PDCD1 gene are chr9:5,450,503-5,470,567 (in GRCh38/hg38) and chr9:5,450,503-5,470,567 (in GRCh37/hg19). The GenBank reference PD1 mRNA sequence is known under accession no. NM_001267706.1, NM_001314029.2 and NM_014143.4. Approved PD-L1-inhibiting antibodies include atezolizumab, avelumab, and durvalumab. PD-L1 siRNA and shRNA products are available through e.g. Origene.

CTLA4

Aliases of CTLA4 provided in GeneCards® include Cytotoxic T-Lymphocyte Associated Protein 4; CTLA-4; CD152; Insulin-Dependent Diabetes Mellitus 12; Cytotoxic T-Lymphocyte Protein 4; Celiac Disease 3; GSE; Ligand And Transmembrane Spliced Cytotoxic T Lymphocyte Associated Antigen 4; Cytotoxic T Lymphocyte Associated Antigen 4 Short Spliced Form; Cytotoxic T-Lymphocyte-Associated Serine Esterase-4; Cytotoxic T-Lymphocyte-Associated Antigen 4; CELIAC3; IDDM12; ALPS5; and GRD4. The genomic locations for the CTLA4 gene are chr2:203,867,771-203,873,965 (in GRCh38/hg38) and chr2:204,732,509-204,738,683 (in GRCh37/hg19). The GenBank reference CTLA4 mRNA sequences are known under accession nos. NM_001037631.3 and NM_005214.5. Approved CTLA4-inhibiting antibodies include ipilumab; CTLA4-inhibiting antibodies under development include tremelimumab. CTLA4 siRNA and shRNA products are available through e.g. Origene.

The term “antagonist” or “inhibitor” of a target as used herein refers to antagonists or inhibitors of function or to antagonists or inhibitors of expression of a target of interest. Antagonists of a target may also be compounds binding to a target (e.g. tumor) cell and causing its killing; examples of such antagonists include e.g. antibody-(cytotoxic) drug-conjugates or antibodies capable of causing ADCC. Interchangeable alternatives for “antagonist” include inhibitor, repressor, suppressor, inactivator, and blocker. An “antagonist” thus refers to a molecule that decreases, blocks, inhibits, abrogates, or interferes with target expression, activation or function.

Downregulating of expression of a gene encoding a target is feasible through gene therapy (e.g., by administering siRNA, shRNA or antisense oligonucleotides to the target gene). Biopharmaceutical and gene therapeutic antagonists include such entities as antisense oligonucleotides, gapmers, siRNA, shRNA, zinc-finger nucleases, meganucleases, TAL effector nucleases, CRISPR-Cas effectors, monoclonal antibodies or fragments thereof, alpha-bodies, nanobodies, intrabodies, aptamers, DARPins, affibodies, affitins, anticalins, monobodies, PROTACs, LYTACs, etc. (general description of these compounds included hereinafter).

Inactivation or inhibition of a process as envisaged in the current invention refers to different possible levels of inactivation or inhibition, e.g., at least 10%, at least 20%, at least 30%, at least 40%, at least 50%, at least 60%, at least 70%, at least 80%, at least 90%, at least 95%, or even 100% of inactivation or inhibition (compared to a normal situation). The nature of the inactivating, inhibiting or antagonizing compound is not vital/essential to the invention as long as the process envisaged is inactivated, inhibited or antagonized such as to treat or inhibit tumor growth or such as to inhibit progression or relapse of tumor growth.

Genetic Inhibition of a Target of Interest

Downregulating expression of a gene encoding a target is feasible through gene therapy or gene therapeutic agents, in particular gene therapeutic antagonist agents. Such agents include such entities as antisense oligonucleotides, gapmers, siRNA, shRNA, zinc-finger nucleases, meganucleases, Argonaute, TAL effector nucleases, CRISPR-Cas effectors, and nucleic acid aptamers. In particular, any of these agents is specifically or exclusively acting on or antagonizing or inhibiting the target of interest; or any of these agents is designed for specifically or exclusively acting on or antagonizing or inhibiting the target of interest.

One process of modulating/downregulating expression of a gene/target gene of interest relies on antisense oligonucleotides (ASOs), or variants thereof such as gapmers. An antisense oligonucleotide (ASO) is a short strand of nucleotides and/or nucleotide analogues that hybridizes with the complementary mRNA in a sequence-specific manner. Formation of the ASO-mRNA complex ultimately results in downregulation of target protein expression (Chan et al. 2006, Clin Exp Pharmacol Physiol 33:533-540; this reference also describes some of the software available for assisting in design of ASOs). Modifications to ASOs can be introduced at one or more levels: phosphate linkage modification (e.g. introduction of one or more of phosphodiester, phosphoramidate or phosphorothioate bonds), sugar modification (e.g. introduction of one or more of LNA (locked nucleic acids), 2′-O-methyl, 2′-O-methoxy-ethyl, 2′-fluoro, S-constrained ethyl or tricyclo-DNA and/or non-ribose modifications (e.g. introduction of one or more of phosphorodiamidate morpholinos or peptide nucleic acids). The introduction of 2′-modifications has been shown to enhance safety and pharmacologic properties of antisense oligonucleotides. Antisense strategies relying on degradation of mRNA by RNase H requires the presence of nucleotides with a free 2′-oxygen, i.e. not all nucleotides in the antisense molecule should be 2′-modified. The gapmer strategy has been developed to this end. A gapmer antisense oligonucleotide consists of a central DNA region (usually a minimum of 7 or 8 nucleotides) with (usually 2 or 3) 2′-modified nucleosides flanking both ends of the central DNA region. This is sufficient for the protection against exonucleases while allowing RNAseH to act on the (2′-modification free) gap region. Antidote strategies are available as demonstrated by administration of an oligonucleotide fully complementary to the antisense oligonucleotide (Crosby et al. 2015, Nucleic Acid Ther 25:297-305).

Another process to modulate expression of a gene/target gene of interest is based on the natural process of RNA interference. It relies on double-stranded RNA (dsRNA) that is cut by an enzyme called Dicer, resulting in double stranded small interfering RNA (siRNA) molecules which are 20-25 nucleotides long. siRNA then binds to the cellular RNA-Induced Silencing Complex (RISC) separating the two strands into the passenger and guide strand. While the passenger strand is degraded, RISC is cleaving mRNA specifically at a site instructed by the guide strand. Destruction of the mRNA prevents production of the protein of interest and the gene is ‘silenced’. siRNAs are dsRNAs with 2 nt 3′ end overhangs whereas shRNAs are dsRNAs that contains a loop structure that is processed to siRNA. shRNAs are introduced into the nuclei of target cells using a vector (e.g. bacterial or viral) that optionally can stably integrate into the genome. Apart from checking for lack of cross-reactivity with non-target genes, manufacturers of RNAi products provide guidelines for designing siRNA/shRNA. siRNA sequences between 19-29 nt are generally the most effective. Sequences longer than 30 nt can result in nonspecific silencing. Ideal sites to target include AA dinucleotides and the 19 nt 3′ of them in the target mRNA sequence. Typically, siRNAs with 3′ dUdU or dTdT dinucleotide overhangs are more effective. Other dinucleotide overhangs could maintain activity but GG overhangs should be avoided. Also to be avoided are siRNA designs with a 4-6 poly(T) tract (acting as a termination signal for RNA pol III), and the G/C content is advised to be between 35-55%. shRNAs should comprise sense and antisense sequences (advised to each be 19-21 nt in length) separated by loop structure, and a 3′ AAAA overhang. Effective loop structures are suggested to be 3-9 nt in length. It is suggested to follow the sense-loop-antisense order in designing the shRNA cassette and to avoid 5′ overhangs in the shRNA construct. shRNAs are usually transcribed from vectors, e.g. driven by the Pol III U6 promoter or H1 promoter. Vectors allow for inducible shRNA expression, e.g. relying on the Tet-on and Tet-off inducible systems commercially available, or on a modified U6 promoter that is induced by the insect hormone ecdysone. A Cre-Lox recombination system has been used to achieve controlled expression in mice. Synthetic shRNAs can be chemically modified to affect their activity and stability. Plasmid DNA or dsRNA can be delivered to a cell by means of transfection (lipid transfection, cationic polymer-based nanoparticles, lipid or cell-penetrating peptide conjugation, lipid nanoparticles or LNPs) or electroporation. Vectors include viral vectors such as lentiviral, retroviral, adenoviral and adeno-associated viral vectors.

Ribozymes (ribonucleic acid enzymes) are another type of molecules that can be used to modulate expression of a gene/target gene of interest. They are RNA molecules capable of catalyzing specific biochemical reactions, in the current context capable of targeted cleavage of nucleotide sequences, in particular targeted cleavage of a RNA/RNA target of interest. Examples of ribozymes include the hammerhead ribozyme, the Varkud Satellite ribozyme, Leadzyme and the hairpin ribozyme.

Besides the use of the inhibitory RNA technology, modulation of expression of a gene of interest can be achieved at DNA level such as by gene therapy to knock-out, knock-down or disrupt the target gene/gene of interest. As used herein, a “gene knock-out” can be a gene knockdown or the gene can be knocked out, knocked down, disrupted or modified by a mutation such as, a point mutation, an insertion, a deletion, a frameshift, or a missense mutation by techniques such as described hereafter, including, but not limited to, retroviral gene transfer. One way in which genes can be knocked out, knocked down, disrupted or modified is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target a desired DNA sequence/DNA sequence of interest, which enable zinc-finger nucleases to target unique sequence within a complex genome. By taking advantage of the endogenous DNA repair machinery, these reagents can be used to precisely alter the genomes of higher organisms.

Other technologies for genome customization that can be used to specifically or selectively knock out, knock down or disrupt a gene/gene of interest are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes) or DNA sequences of interest. Another recent genome editing technology is the CRISPR/Cas system, which can be used to achieve RNA-guided genome engineering (including knock-out, knock-down or disruption of a gene of interest). CRISPR interference is a genetic technique which allows for sequence-specific control of expression of a gene of interest in prokaryotic and eukaryotic cells. It is based on the bacterial immune system-derived CRISPR (clustered regularly interspaced palindromic repeats) pathway. Recently, it was demonstrated that the CRISPR-Cas editing system can also be used to target RNA. It has been shown that the Class 2 type VI-A CRISPR-Cas effector C2c2 (Cas13a; CRISPR-Cas13a or CRISPR-C2c2) can be programmed to cleave single stranded RNA targets carrying complementary protospacers (Abudayyeh et al. 2016 Science353/science.aaf5573). C2c2 is a single-effector endoRNase mediating ssRNA cleavage once it has been guided by a single crRNA guide toward a target RNA/RNA of interest.

Methods for administering nucleic acids include methods applying non-viral (DNA or RNA) or viral nucleic acids (DNA or RNA viral vectors). Methods for non-viral gene therapy include the injection of naked DNA (circular or linear), electroporation, the gene gun, sonoporation, magnetofection, the use of oligonucleotides, lipoplexes (e.g. complexes of nucleic acid with DOTAP or DOPE or combinations thereof, complexes with other cationic lipids), dendrimers, viral-like particles, inorganic nanoparticles, hydrodynamic delivery, photochemical internalization (Berg et al. 2010, Methods Mol Biol 635:133-145) or combinations thereof.

Many different vectors have been used in human nucleic acid therapy trials and a listing can be found on http://www.abedia.com/wiley/vectors.php. Currently the major groups are adenovirus or adeno-associated virus vectors (in about 21% and 7% of the clinical trials), retrovirus vectors (about 19% of clinical trials), naked or plasmid DNA (about 17% of clinical trials), and lentivirus vectors (about 6% of clinical trials). Combinations are also possible, e.g. naked or plasmid DNA combined with adenovirus, or RNA combined with naked or plasmid DNA to list just a few. Other viruses (e.g. alphaviruses, vaccinia viruses such as vaccinia virus Ankara) are used in nucleic acid therapy and are not excluded in the context of the current invention.

Administration may be aided by specific formulation of the nucleic acid e.g. in liposomes (lipoplexes) or polymersomes (synthetic variants of liposomes), in lipid nanoparticles (LNPs), as polyplexes (nucleic acid complexed with polymers), carried on dendrimers, in inorganic (nano)particles (e.g. containing iron oxide in case of magnetofection), or combined with a cell penetrating peptide (CPP) to increase cellular uptake. Organ- or cellular-targeting strategies may also be applied to the nucleic acid (nucleic acid combined with organ- or cell-targeting moiety); these include passive targeting (mostly achieved by adapted formulation) or active targeting (e.g. by coupling a nucleic acid-comprising nanoparticle with any compound (e.g. an aptamer or antibody or antigen binding molecule) binding to a target organ- or cell-specific antigen) (e.g. Steichen et al. 2013, Eur J Pharm Sci 48:416-427).

CPPs enable translocation of the drug of interest coupled to them across the plasma membrane. CPPs are alternatively termed Protein Transduction Domains (TPDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia—Antp), pVEC (derived from murine vascular endothelial cadherin), signal-sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein. CPPs can be coupled to carriers such as nanoparticles, liposomes, micelles, or generally any hydrophobic particle. Coupling can be by absorption or chemical bonding, such as via a spacer between the CPP and the carrier. To increase target specificity an antibody binding to a target-specific antigen can further be coupled to the carrier (Torchilin 2008, Adv Drug Deliv Rev 60:548-558). CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8:e71752).

Any other modification of the DNA or RNA to enhance efficacy of nucleic acid therapy is likewise envisaged to be useful in the context of the applications of a nucleic acid as outlined herein. The enhanced efficacy can reside in enhanced expression, enhanced delivery properties, enhanced stability and the like. The applications of a nucleic acid as outlined herein may thus rely on using a modified nucleic acid as described above. Further modifications of the nucleic acid may include those suppressing inflammatory responses (hypoinflammatory nucleic acids).

Pharmacological Inhibition of a Target of Interest

Pharmacological inhibition in general occurs by means of an agent inhibiting at least one of the biological activities (if more than one is known) of a target protein of interest. In particular, such pharmacological inhibitor is binding, such as specifically and/or exclusively binding to a target protein or protein of interest, and/or is specifically and/or exclusively inhibiting the targeted biological activity of the a target protein of interest.

Such binding may occur with high affinity although this is not an absolute requirement. The pharmacological inhibitor of a target protein or protein of interest may for instance have a binding affinity (dissociation constant) to (one of) its target of about 1000 nM or less, a binding affinity of about 100 nM or less, a binding affinity of about 50 nM or less, a binding affinity of about 10 nM or less, or a binding affinity of about 1 nM or less. Cross-reactivity of a pharmacological inhibitor with more than one protein is possible; for clinical development it can e.g. be desired to be able to test a pharmacological inhibitor in a suitable in vitro or in vivo animal model before starting clinical testing with the same pharmacological inhibitor in a human population, which requires the pharmacological inhibitor to cross-react with the animal (or other non-human) target protein and with the orthologous human target protein (orthologous proteins are homologous proteins separated by a speciation event).

Specificity of binding refers to the situation in which a pharmacological inhibitor is, at a certain concentration (sufficient to inhibit the target protein or protein of interest) binding to the target protein with higher affinity (e.g. at least 2-fold, 5-fold, or at least 10-fold higher affinity, e.g. at least 20-, 50- or 100-fold or more higher affinity) than the affinity with which it is possibly (if at all) binding to other proteins (proteins not of interest). Such specificity of binding is in particular determined within the setting of the target subject (e.g. human patient, or animal model) and thus can encompass/does not exclude binding to (at least one) orthologous target proteins. Exclusivity of binding refers to the situation in which a pharmacological inhibitor is binding only to the target protein of interest (and possibly to (at least one) orthologous target protein).

Alternatively, the pharmacological inhibitor may exert the desired level of inhibition of the targeted biological activity or biological activity of interest of a target protein or protein of interest with an IC50 of 1000 nM or less, with an IC50 of 500 nM or less, with an IC50 of 100 nM or less, with an IC50 of 50 nM or less, with an IC50 of 10 nM or less, or with an IC50 of 1 nM or less.

Cross-inhibition by a pharmacological inhibitor of more than one protein is possible; for clinical development it can e.g. be desired to be able to test a pharmacological inhibitor in a suitable in vitro or in vivo animal model before starting clinical testing with the same pharmacological inhibitor in a human population, which requires the pharmacological inhibitor to cross-inhibit the animal (or other non-human) target protein and the orthologous human target protein.

Specificity of inhibition refers to the situation in which a pharmacological inhibitor is, at a certain concentration (sufficient to inhibit the target protein or protein of interest) inhibiting the target protein with higher efficacy (e.g. with an at least 2-fold, 5-fold, or 10-fold lower IC50, e.g. at least 20-, 50- or 100-fold or more lower IC50) than the efficacy with which it is possibly (if at all) inhibiting other proteins (proteins not of interest). Such specificity of inhibition is in particular determined within the setting of the target subject (e.g. human patient, or animal model) and thus can encompass/does not exclude inhibition of (at least one) orthologous target proteins. Exclusivity of inhibition refers to the situation in which a pharmacological inhibitor is inhibiting only the target protein of interest (or (at least one) orthologous target protein).

Specificity of inhibition may refer to inhibition of a single biological activity of a protein of interest (and possibly of (at least one) orthologue) if the protein of interest is known to have more than one biological activity; or may refer to inhibition of the protein of interest (and possibly of (at least one) orthologue) as such, independent of it possibly having multiple biological activities.

Exclusivity of inhibition refers to the situation in which a pharmacological inhibitor is inhibiting only a single biological activity of a protein of interest (and possibly of (at least one) orthologue) if the protein of interest is known to have more than one biological activity; or may refer to inhibition of only the protein of interest (and possibly of (at least one) orthologue) as such, independent of it possibly having multiple biological activities.

In general, the agent inhibiting a target protein or protein of interest is a polypeptide, a polypeptidic agent, an aptamer, or a combination of any of the foregoing. Examples of such pharmacologic inhibitors, all specifically and/or exclusively binding to and/or inhibiting the target protein of interest include immunoglobulin variable domains, antibodies (in particular monoclonal antibodies) or a fragment thereof, alpha-bodies, nanobodies, intrabodies, aptamers, DARPins, affibodies, affitins, anticalins, monobodies, and bicyclic peptides.

The term “antibody” as used herein, refers to an immunoglobulin (Ig) molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The term “immunoglobulin domain” as used herein refers to a globular region of an antibody chain (such as e.g., a chain of a conventional 4-chain antibody or a chain of a heavy chain antibody), or to a polypeptide that essentially consists of such a globular region/immunoglobulin domain. Immunoglobulin domains are characterized in that they retain the immunoglobulin fold characteristic of antibody molecules, which consists of a two-layer sandwich of about seven antiparallel β-strands arranged in two β-sheets, optionally stabilized by a conserved disulphide bond.

The specificity of an antibody/immunoglobulin/immunoglobulin domain/immunoglobulin variable domain (IVD) for an antigen is defined by the composition of the antigen-binding domains in the antibody/immunoglobulin/IVD (usually one or more of the CDRs, the particular amino acids of the antibody/immunoglobulin/IVD interacting with the antigen, and forming the paratope or antigen-binding site) and the composition of the antigen (the parts of the antigen interacting with the antibody/immunoglobulin/IVD and forming the epitope or antibody binding site). Specificity of binding is understood to refer to a binding between an antibody/immunoglobulin/IVD with a single target molecule or with a limited number of target molecules that (happen to) share an epitope recognized by the antibody/immunoglobulin/IVD.

Affinity of an antibody/immunoglobulin/IVD for its target is a measure for the strength of interaction between an epitope on the target (antigen) and an epitope/antigen binding site in the antibody/immunoglobulin/IVD. It can be defined as:

$${K}_{A} = \frac{[ Ab - Ag ]}{[ Ab ] [ Ag ]}$$

Wherein KA is the affinity constant, [Ab] is the molar concentration of unoccupied binding sites on the antibody/immunoglobulin/IVD, [Ag] is the molar concentration of unoccupied binding sites on the antigen, and [Ab-Ag] is the molar concentration of the antibody-antigen complex. Avidity provides information on the overall strength of an antibody/immunoglobulin/IVD-antigen complex, and generally depends on the above-described affinity, the valency of antibody/immunoglobulin/IVD and of antigen, and the structural interaction of the binding partners.

The term “immunoglobulin variable domain” (abbreviated as “IVD”) as used herein means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Methods for delineating/confining a CDR in an antibody/immunoglobulin/immunoglobulin domain/IVD have been described in the art and include the Kabat, Chothia, IMTG, Martin, Gelfand, and Honneger systems (see Dondelinger et al. 2018, Front Immunol 9:2278).

The term “immunoglobulin single variable domain” (abbreviated as “ISVD”), equivalent to the term “single variable domain”, defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associated) immunoglobulin domains such as light and heavy chain variable domains, i.e., by a VH-VL pair of immunoglobulin domains, which jointly bind to an epitope of the respective antigen. In contrast, immunoglobulin single variable domains are capable of specifically binding to an epitope of the antigen without pairing with an additional immunoglobulin variable domain. The binding site of an immunoglobulin single variable domain is formed by a single VH/VHH or VL domain. Hence, the antigen binding site of an immunoglobulin single variable domain is formed by no more than three CDRs. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody® (as defined herein, and including but not limited to a VHH); other single variable domains, or any suitable fragment of any one thereof. In particular, the immunoglobulin single variable domain may be a Nanobody® (as defined herein) or a suitable fragment thereof. Note: Nanobody®, Nanobodies® and Nanoclone® are registered trademarks of Ablynx (now part of Sanofi). For a general description of Na nobodies®, reference is made to the further description below, as well as to e.g. WO2008/020079. “VHH domains”, also known as VHHs, VHH domains, VHH antibody fragments, and VHH antibodies, have originally been described as the antigen binding immunoglobulin (variable) domain of “heavy chain antibodies” (i.e., of “antibodies devoid of light chains”; Hamers-Casterman et al. 1993, Nature 363:446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody®, reference is made to the review article by Muyldermans 2001 (Rev Mol Biotechnol 74:277-302), as well as to the following patent applications, which are mentioned as general background art: WO 94/04678, WO 95/04079, WO 96/34103; WO 94/25591, WO 99/37681, WO 00/40968, WO 00/43507, WO 00/65057, WO 01/40310, WO 01/44301, EP 1134231 and WO 02/48193; WO 97/49805, WO 01/21817, WO 03/035694, WO 03/054016 and WO 03/055527; WO 03/050531; WO 01/90190; WO 03/025020; WO 04/041867, WO 04/041862, WO 04/041865, WO 04/041863, WO 04/062551, WO 05/044858, WO 06/40153, WO 06/079372, WO 06/122786, WO 06/122787 and WO 06/122825. As described in these references, Nanobody® (in particular VHH sequences and partially humanized Nanobody®) can in particular be characterized by the presence of one or more “hallmark residues” in one or more of the framework sequences. A further description of the Nanobody®, including humanization and/or camelization of Nanobody®, as well as other modifications, parts or fragments, derivatives or “Nanobody® fusions”, multivalent constructs (including some non-limiting examples of linker sequences) and different modifications to increase the half-life of the Nanobody® and their preparations can be found in e.g. WO 08/101985 and WO 08/142164.

“Domain antibodies”, also known as “Dabs” (the terms “Domain Antibodies” and “dAbs” being used as trademarks by the GlaxoSmithKline group of companies) have been described in e.g., EP 0368684, Ward et al. 1989 (Nature 341:544-546), Holt et al. 2003 (Trends in Biotechnology 21:484-490) and WO 03/002609, WO 04/068820, WO 06/030220, and WO 06/003388. Domain antibodies essentially correspond to the VH or VL domains of non-camelid mammalians, in particular human 4-chain antibodies. In order to bind an epitope as a single antigen binding domain, i.e., without being paired with a VL or VH domain, respectively, specific selection for such antigen binding properties is required, e.g. by using libraries of human single VH or VL domain sequences. Domain antibodies have, like VHHs, a molecular weight of approximately 13 to approximately 16 kDa and, if derived from fully human sequences, do not require humanization for e.g. therapeutic use in humans. It should also be noted that single variable domains can be derived from certain species of shark (for example, the so-called “IgNAR domains”, see e.g. WO 05/18629).

Alphabodies are also known as Cell-Penetrating Alphabodies and are small 10 kDa proteins engineered to bind to a variety of antigens.

Aptamers have been selected against small molecules, toxins, peptides, proteins, viruses, bacteria, and even against whole cells. DNA/RNA/XNA aptamers are single stranded oligonucleotides and are typically around 15-60 nucleotides in length, although longer sequences of 220nt have been selected; they can contain non-natural nucleotides (XNA) as described for antisense RNA. A nucleotide aptamer binding to the vascular endothelial growth factor (VEGF) was approved by FDA for treatment of macular degeneration. Variants of RNA aptamers are spiegelmers are composed entirely of an unnatural L-ribonucleic acid backbone. A Spiegelmer of the same sequence has the same binding properties of the corresponding RNA aptamer, except it binds to the mirror image of its target molecule.

Peptide aptamers consist of one (or more) short variable peptide domains, attached at both ends to a protein scaffold, e.g. the Affimer scaffold based on the cystatin protein fold. Although not called aptamers, a type of further variation is described in e.g. WO 2004/077062 wherein e.g. 2 peptide loops are attached to an organic scaffold to arrive at a bicyclic peptide (which can be further multimerized). Phage-display screening of such bicyclic peptides to arrive at species binding with high-affinity to a target has proven to be possible in e.g. WO 2009/098450.

DARPins stands for designed ankyrin repeat proteins. DARPin libraries with randomized potential target interaction residues, with diversities of over 10″12 variants, have been generated at the DNA level. From these, DARPins can be selected for binding to a target of choice with picomolar affinity and specificity. Affitins, or nanofitins, are artificial proteins structurally derived from the DNA binding protein Sac7d, found in Sulfolobus acidocaldarius. By randomizing the amino acids on the binding surface of Sac7d and subjecting the resulting protein library to rounds of ribosome display, the affinity can be directed towards various targets, such as peptides, proteins, viruses, and bacteria.

Anticalins are derived from human lipocalins which are a family of naturally binding proteins and mutation of amino acids at the binding site allows for changing the affinity and selectivity towards a target of interest. They have better tissue penetration than antibodies and are stable at temperatures up to 70° C.

Monobodies are synthetic binding proteins that are constructed starting from the fibronectin type III domain (FN3) as a molecular scaffold.

Affibodies are composed of alpha helices and lack disulfide bridges, and are based on the Z or IgG-binding domain scaffold of protein A wherein amino acids located in the parental binding domain are randomized. Screening for affibodies for specific binding to a desired target typically is performed using phage display.

Intrabodies are antibodies binding and/or acting to intracellular target; this typically requires the expression of the antibody within the target cell, which can be accomplished by gene therapy/genetic modification involving introduction in a cell of a suitable genetic construct or vector comprising a suitable promoter (e.g. inducible, organ- or cell-specific, . . . ) operably linked to an intrabody coding sequence.

Pharmacological Knock-Down of a Protein of Interest

Several technologies can be applied to cause pharmacological knock-down of a target protein or protein of interest. Outlined hereafter are the general principles of agents causing pharmacological knock-down of a target protein by means of inducing (proteolytic) degradation of that target protein. A proteolysis targeting chimera, or PROTAC, is a chimeric polypeptidic molecule comprising a moiety recognized by an ubiquitin ligase and a moiety binding to a target protein. Interaction of the PROTAC with the target protein causes it to be poly-ubiquinated followed by proteolytic degradation by a cell's own proteasome. As such, a PROTAC provides the possibility of pharmacologically knocking down a target protein. The moiety binding to a target protein can be a peptide or a small molecule (reviewed in, e.g., Zou et al. 2019, Cell Biochem Funct 37:21-30). Other such target protein degradation inducing technologies include dTAG (degradation tag; see, e.g., Nabet et al. 2018, Nat Chem Biol 14:431), Trim-Away (Clift et al. 2017, Cell 171:1692-1706), chaperone-mediated autophagy targeting (Fan et al. 2014, Nat Neurosci 17:471-480) and SNIPER (specific and non-genetic inhibitor of apoptosis protein (IAP)-dependent protein erasers; Naito et al. 2019, Drug Discov Today Technol, doi:10.1016/j.ddtec.2018.12.002).

Lysosome targeting chimeras, or LYTACs, are chimeric molecules comprising a moiety binding to a lysosomal targeting receptor (LTR) and a moiety binding to a target protein (such as an antibody). Interaction of the LYTAC with the target protein causes it to be internalized followed by lysosomal degradation. A prototypic LTR is the cation-independent mannose-6-phosphate receptor (ciMPR) and an LTR binding moiety is e.g. an agonist glycopeptide ligand of ciMPR. The target protein can be a secreted protein or a membrane protein (see, e.g., Banik et al. 2019, doi.org/10.26434/chemrxiv.7927061.v1).

Treatment/Therapeutically Effective Amount

The terms therapeutic modality, therapeutic agent, and agent are used interchangeably herein. All refer to a therapeutically active compound, to a combination of therapeutically active compounds, or to a therapeutically active composition (comprising one or more therapeutically active compounds). “Treatment”/“treating” refers to any rate of reduction, delaying or retardation of the progress of the disease or disorder, or a single symptom thereof, compared to the progress or expected progress of the disease or disorder, or single symptom thereof, when left untreated. This implies that a therapeutic modality on its own may not result in a complete or partial response (or may even not result in any response), but may, in particular when combined with other therapeutic modalities (such as, but not limited thereto: surgery, radiation, etc.), contribute to a complete or partial response (e.g. by rendering the disease or disorder more sensitive to therapy). More desirable, the treatment results in no/zero progress of the disease or disorder, or single symptom thereof (i.e. “inhibition” or “inhibition of progression”), or even in any rate of regression of the already developed disease or disorder, or single symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. by patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment. The time-frame over which the improvement is evaluated will depend on the type of criteria/disease observed and can be determined by the person skilled in the art.

A “therapeutically effective amount” refers to an amount of a therapeutic agent to treat or prevent a disease or disorder in a subject (such as a mammal). In the case of cancers, the therapeutically effective amount of the therapeutic agent is treating cancer, i.e., it may reduce the number of cancer cells; reduce the primary tumor size; inhibit (i.e., slow down to some extent and preferably stop) cancer cell infiltration into peripheral organs; inhibit (i.e., slow down to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, e.g., be measured by assessing the duration of survival (e.g. overall survival), time to disease progression (TTP), response rates (e.g., complete response and partial response, stable disease), length of progression-free survival (PFS), duration of response, and/or quality of life.

The term “effective amount” or “therapeutically effective amount” refers to the dosing regimen of the agent/therapeutic agent or composition comprising the agent/therapeutic agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects (often expressed as maximum tolerable dose, MTD). To obtain or maintain the effective amount, the agent or composition comprising the agent may be administered as a single dose or in multiple doses. The effective amount may further vary depending on the severity of the condition that needs to be treated; this may depend on the overall health and physical condition of the subject or patient and usually the treating doctor's or physician's assessment will be required to establish what is the effective amount. The effective amount may further be obtained by a combination of different types of contacting or administration.

The aspects and embodiments described above in general may comprise the administration of one or more therapeutic compounds to a subject (such as a mammal) in need thereof, i.e., harboring a tumor, cancer or neoplasm in need of treatment. In general a (therapeutically) effective amount of (a) therapeutic compound(s) is administered to the mammal in need thereof in order to obtain the described clinical response(s).

“Administering” means any mode of contacting that results in interaction between an agent (e.g. a therapeutic compound) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

The invention further relates to a combination of a P2Y6 inhibitor and an immune checkpoint inhibitor. Alternatively, the invention relates to a combination of a composition, such as a pharmaceutically acceptable composition, comprising a P2Y6 inhibitor and a composition, such as a pharmaceutically acceptable composition, comprising an immune checkpoint inhibitor. In one embodiment, the combination of a P2Y6 inhibitor and an immune checkpoint inhibitor is for use as a medicament.

In yet a further aspect, the invention relates to isolated macrophages wherein the expression of the P2Y6 gene is knocked-out or otherwise downregulated (see above), such as by conditionally expressing a P2Y6 inhibitor. In one embodiment, such isolated P2Y6 knock-out macrophage, or such isolated macrophage conditionally expressing a P2Y6 inhibitor is for use as a medicament, for use in the manufacture of a medicament, for use in treating cancer or a tumor, for application in a method of treating cancer or a tumor, for use in inhibiting progression of a cancer or a tumor, or of application in a method inhibiting progression of a cancer or tumor.

The invention further relates to pharmaceutical compositions comprising an isolated P2Y6 knock-out macrophage or an isolated macrophage conditionally expressing a P2Y6 inhibitor, and further comprising an excipient.

The invention further relates to combination of an P2Y6 knock-out macrophage and an immune checkpoint inhibitor, or a combination of an isolated macrophage conditionally expressing a P2Y6 inhibitor and an immune checkpoint inhibitor. In one embodiment, such combination is for use as a medicament, for use in the manufacture of a medicament, for use in treating cancer or a tumor, for application in a method of treating cancer or a tumor, for use in inhibiting progression of a cancer or a tumor, or of application in a method inhibiting progression of a cancer or tumor.

In yet a further aspect, the invention provides macrophages wherein the expression of the P2Y6 gene is knocked-out or otherwise downregulated (see above) as inhibitor of P2Y6 for use in any of the above-described aspects. In particular, such macrophages (isolated P2Y6 knock out macrophages, or isolated macrophages conditionally expressing a P2Y6 inhibitor) are for use in a treatment (therapeutic or prophylactic) comprising transfer or adoptive transfer of the macrophages to a subject.

“Combination”, “combination in any way” or “combination in any appropriate way” as referred to herein is meant to refer to any sequence of administration of two (or more) therapeutic modalities, i.e. the administration of the two (or more) therapeutic modalities can occur concurrently in time or separated from each other for any amount of time; and/or “combination”, “combination in any way” or “combination in any appropriate way” as referred to herein can refer to the combined or separate formulation of the two (or more) therapeutic modalities, i.e. the two (or more) therapeutic modalities can be individually provided in separate vials or (other suitable) containers, or can be provided combined in the same vial or (other suitable) container. When combined in the same vial or (other suitable) container, the two (or more) therapeutic modalities can each be provided in the same vial/container chamber of a single-chamber vial/container or in the same vial/container chamber of a multi-chamber vial/container; or can each be provided in a separate vial/container chamber of a multi-chamber vial/container.

The invention further relates to kits comprising a container or vial (any suitable container or vial, such as a pharmaceutically acceptable container or vial) comprising a P2Y6 inhibitor or comprising a composition comprising a P2Y6 inhibitor; and comprising a container or vial (any suitable container or vial, such as a pharmaceutically acceptable container or vial) comprising an immune checkpoint inhibitor or a composition comprising an immune checkpoint inhibitor.

Alternatively, such kits are comprising a container or vial (any suitable container or vial, such as a pharmaceutically acceptable container or vial) comprising a combination of a P2Y6 inhibitor and an immune checkpoint inhibitor (see discussion on “combination in any way” on how such combination in a single container, e.g., vial can be defined).

Other optional components of such kit include one or more diagnostic agents capable of predicting or determining the success of a therapy comprising a combination therapy according to the invention; use instructions; one or more containers with sterile pharmaceutically acceptable carriers, excipients or diluents [such as for producing or formulating a (pharmaceutically acceptable) composition of the invention]; one or more syringes; one or more needles; etc. In particular, such kits may be pharmaceutical kits.

### EXAMPLES

### Example 1. P2Y6 expression

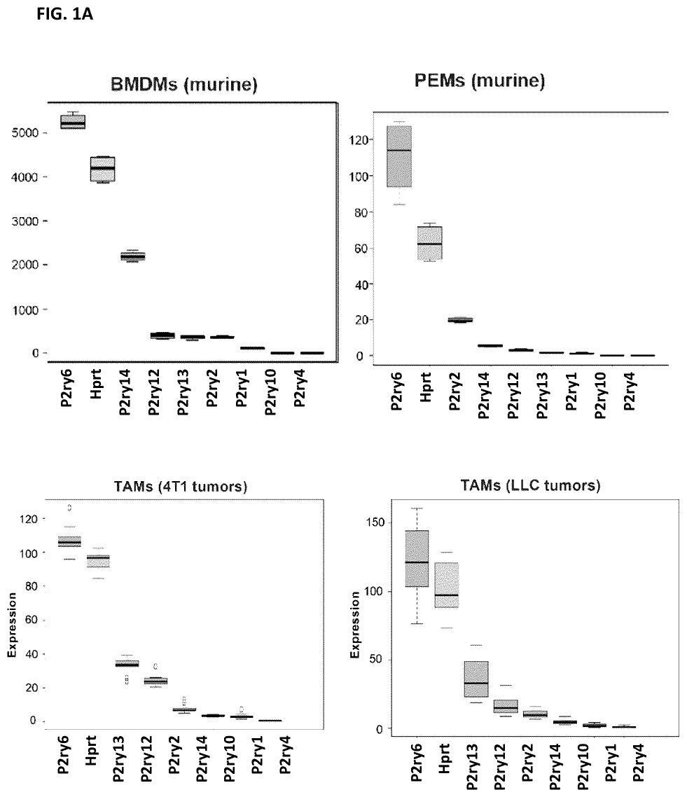

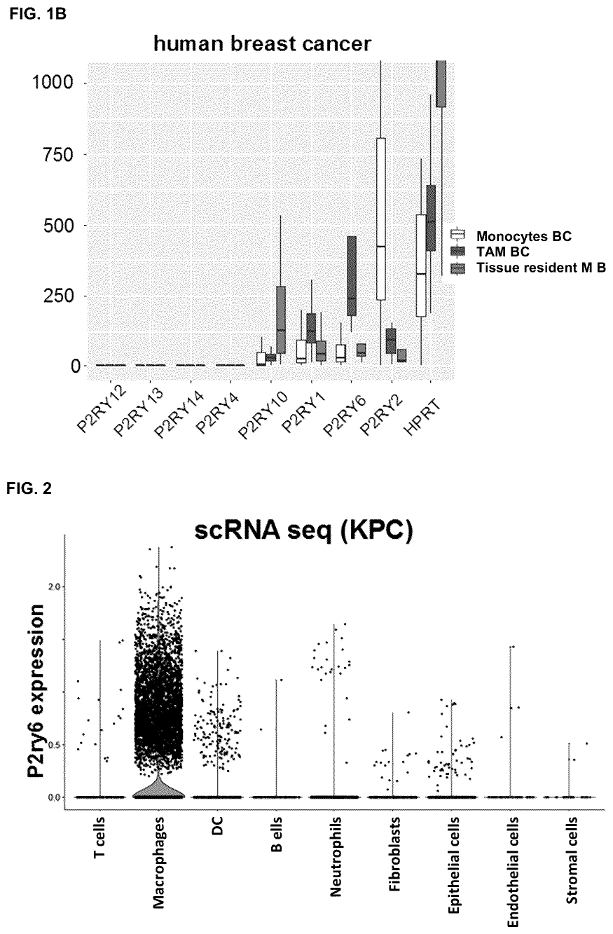

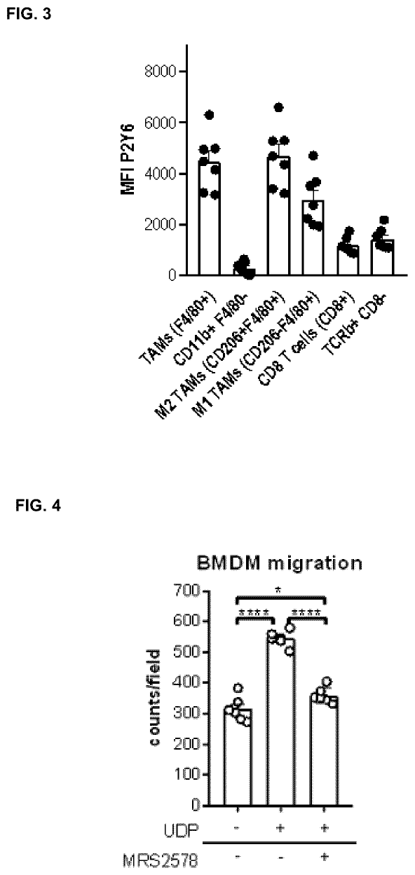

In the P2Y receptor family, P2Y6 was the most represented in bone marrow derived macrophages (BMDMs), peritoneal exudate macrophages (PEMs) and TAMs of several murine and human tumor types (FIGS. 1A and 1B). Moreover, in the tumor microenvironment of both the KPC and Panc02 murine pancreatic cancer model, P2Y6 receptors were highly expressed by macrophages but less in other cells (FIG. 2, 3).

### Example 2. P2Y6 influences macrophage polarization and mediates resistance to α-PD-1 therapy

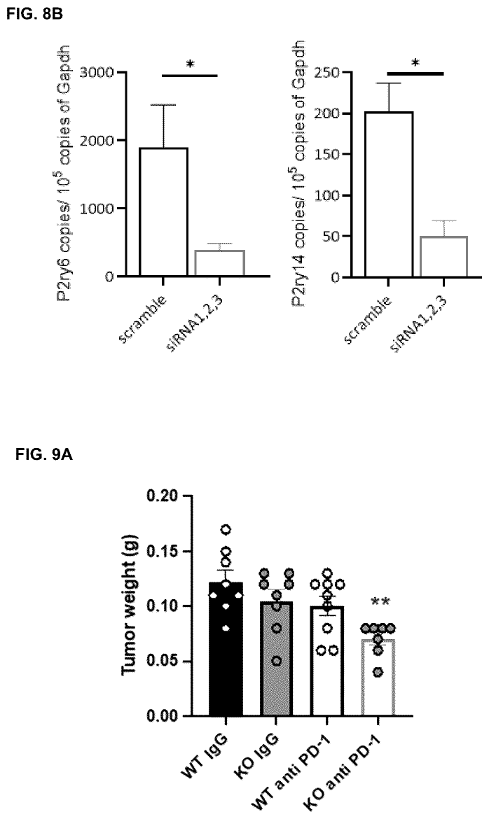

To support the relevance of P2Y6 in macrophage recruitment and phenotypic skewing, several in vitro assays were performed. Firstly, we proved that, in general, BMDMs were migrating towards a gradient of UDP and this migration was specifically inhibited in the presence of the P2Y6 inhibitor MRS 2578 or by siRNA-mediated knockdown of P2Y6 (FIGS. 4, and FIGS. 8A-8B); this migration was not inhibited by sirRNA-mediated knockdown of P2Y14 (FIG. 8A-8B) although P2Y14 is also abundantly expressed in BMDMs (see FIGS. 1A and 1B). P2Y6 receptor expression in macrophages is increased upon stimulation with immunosuppressive cytokines (IL-10) and decreased upon stimulation with immunostimulatory cytokines (IFNγ) (results not shown).

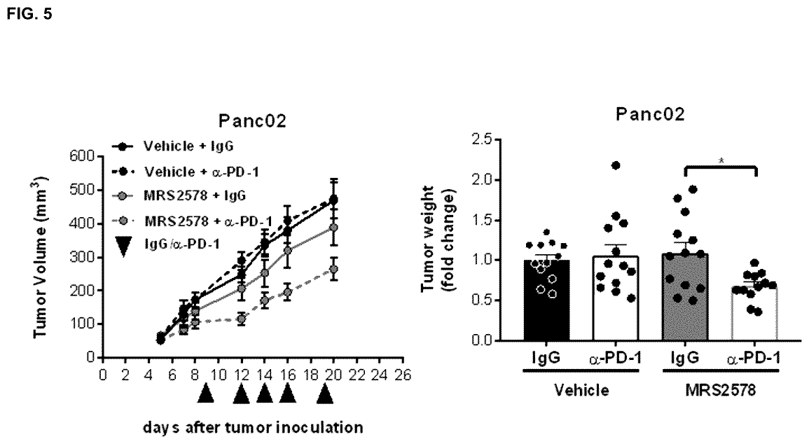

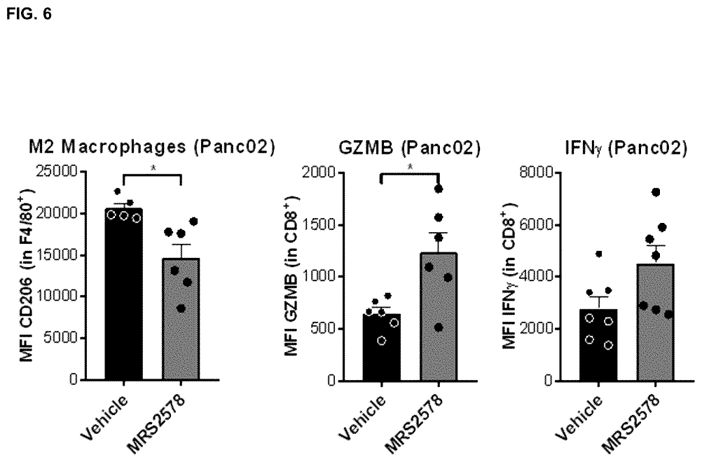

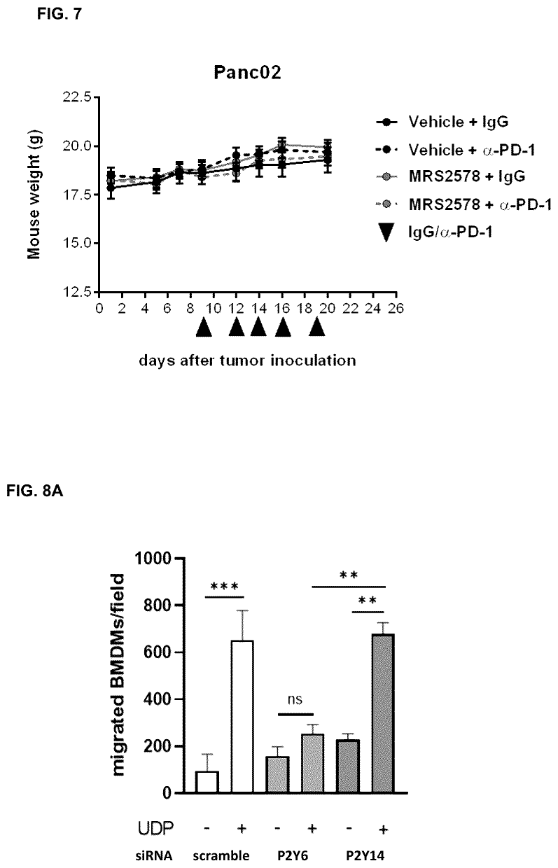

Finally, the essential role of the UPD receptor P2Y6 on macrophages in the resistance to immunotherapy could be verified in vivo using subcutaneously (sc) implanted Panc02 tumors treated with or without the selective P2Y6 receptor antagonist MRS2578. MRS2578 treatment showed no harmful effect on mice (FIG. 7). Treatment with MRS2578 showed a decrease in tumor volume and tumor weight when combined to α-PD-1 therapy (FIG. 5). Correspondingly, CD8+ T cells in the tumors were more activated (GZMB) whereas macrophages showed a reduction of M2 polarization (as measured by expression of CD206 determined by FACS) (FIG. 6). Reduced infiltration of tumor-associated macrophages (TAMs) and reduced infiltration of M2 polarized TAMS into Panc02 tumors was observed as well. Inhibition of the P2Y6 in pancreatic cancer using drugs such as MRS2578 hold the potential to improve pancreatic cancer treatment alone or in combination with checkpoint inhibitors such as anti-PD-1, by improving cytotoxic T cell infiltration.

Expression of P2Y6 in TAMs appears to install a cross-talk between cancer cells and macrophages in order to sustain immunosuppression and to halt T cell functions even in the presence of activatory signals such as immune checkpoint inhibitors. When this cross-talk is broken either by P2Y6R inhibition, the tumor gets infiltrated by effector T cells and becomes T-cell inflamed or “hot”—a condition that enables the efficacy of immune checkpoint inhibitors such as anti-PD1/resistance to such immunotherapy is overcome with the help of P2Y6R inhibition.

From a clinical point of view, pharmacological inhibitors of P2Y6R sensitize PDAC to anti-PD1-based immunotherapy. Such combinations are suitable for the treatment of e.g. patients with unresectable PDAC or with resectable tumors prior to, or after surgery to reduce tumor mass, but also to minimize the risk of relapse.

Retrospective analysis of terminated clinical trials and new prospective studies will tell us whether CDA expression in cancer cells (or alternatively, high UDP levels or high density of P2Y6R⁺ TAMs) might be used as an exclusion criteria when enrolling PDAC patients for cancer immunotherapy.

### Example 3. Adoptive transfer of macrophages engineered toward P2Y6 inhibition

Bone marrow derived macrophages are maintained in culture, optionally in the presence of a pharmacologic P2Y6-inhibitor. Macrophages are then genetically engineered or re-directed such as to knock out the P2Y6 gene, or to introduce a vector or other genetic construct comprising an inducible promotor operably linked to a cassette allowing expression of a genetic or nucleotide based P2Y6-inhibitor (e.g. miRNA, shRNA, antisense RNA, ribozyme). In the latter case the macrophages are conditionally expressing a P2Y6 inhibitor. The engineered macrophages are subsequently transferred into the subject, such as in the subject's muscle(s) or intravenously, such as to treat cancer as described hereinabove. In case of macrophages engineered towards inducible P2Y6-inhibition, the expression inducing compound is administered at an appropriate timepoint to the subject having received the engineered macrophages. The transfer (adoptive cell transfer) can be autologous or heterologous. Adoptive macrophage transfer has been described in the literature (e.g. Ma et al. 2015, Brain Behaviour Immunity 45:157-170; Parsa et al. 2012, Diabetes 61:2881-2892; Wang et al. 2007, Kidney Int 72:290-299; Zhang et al. 2014, Glia 62:804-817).

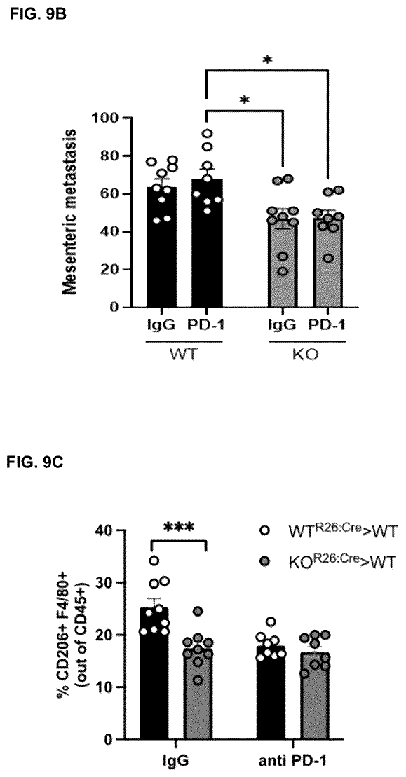

Alternatively, conditional P2Y6 knock out can be achieved using floxing by adoptive transfer of macrophages carrying a floxed P2ry6 gene. C56BL/6 female recipient mice were irradiated with 9.5 Gy. Subsequently, 0.95×10⁷ BM cells from P2ry6^{flox/flox}; Rosa26 Cre/+or P2ry6^{flox/flox}; Rosa26+1+ female mice were injected intravenously in the tail vein. After 5 weeks, P2RY6 deletion was obtained by i.p. injection of tamoxifen (1 mg/mouse/day) for 5 days before orthotopic implantation of 0.5×10⁵ (KPC) FC1245 cancer cells in the head of the pancreas in 20 μL of PBS. At 6, 8 and 10 days after tumor injection, mice were treated intraperitoneally (i.p.) with 10 mg/kg of anti-PD-1 (BioXcell) or control IgG from rat serum (Sigma-Aldrich). Tumor-bearing mice were sacrificed by cervical dislocation at day 13 after tumor injection. Tumors were processed and FACS analysis was performed as described herein. FIG. 9A illustrates that induced P2Y6 knock out in conjunction with anti-PD-1 treatment significantly slows tumor growth. Furthermore, induced P2Y6 knock out alone reduced mesenteric metastasis (FIG. 9B) and resulted in reduced M2 polarization (less CD206) in macrophages (FIG. 9C). Thus, these results confirm results obtained with the pharmacological inhibitor MRS2578 (see Example 2), and overall support the concept of adoptive transfer of macrophage engineered towards P2Y6 inhibition.

### Example 4. Materials & Methods

Animals

C57BL/6 and NMRI-Foxn1^{nu} mice were purchased from Envigo. All mice used were females between 8 and 10 weeks old. Housing and all experimental animal procedures were approved by the Institutional Animal Care and Research Advisory Committee of the KU Leuven (ECD 226/2017).

Cell Lines

The murine pancreatic ductal adenocarcinoma Panc02 cell line was kindly provided by Prof. B. Wiedenmann (Charité, Berlin). Panc02 cells were cultured in DMEM medium (Gibco) supplemented with 10% Fetal Bovine Serum (FBS, Gibco) and 1% (v/v) penicillin-streptomycin solution (Pen/Strep, Gibco). The murine pancreatic FC1245 cell line (hereinafter referred to as KPC or KPC1245), was generated from the KPC murine model (Kras^{LSL.G12D/+}; p53^{R172H/+}; Pdx:Cre^{Tg/+}), and kindly provided by Prof. D. Tuveson (New York, USA). KPC cells were cultured in DMEM medium supplemented with 10% FBS, 1 mM sodium pyruvate (Gibco) and 1% Pen/Strep.

The cells were incubated at 37° C. in a 5% CO₂ humidified atmosphere and subcultured approximately every three days and maintained in a log growth phase.

Tumor Models

4×10⁶ Panc02 cells were injected subcutaneously (s.c.) in the right flank of the mouse in a final suspension of 200 μI PBS. Tumor volumes were measured at least three times per week with a calliper and calculated using the formula: Volume (mm³): ((Width(mm))²×Length(mm))/2. 0.1×10⁶ KPC1245 cells were injected orthotopically into the head of the pancreas in 20 μl of PBS.

Mice were randomized and treated, at indicated time points, intraperitoneally (i.p.) with 10 mg/kg of α-PD-1 (BioXcell) or control IgG from rat serum (Sigma-Aldrich), 3 mg/kg/day MRS2578 (Selleck Chemicals) or control vehicle. At the end stage, tumor weight was measured.

Mice were monitored and weighed continuously during the experiments. Mice showing symptoms of illness, that lost 20% of initial body weight, peritoneal leakage or with ulcerated tumors were sacrificed and excluded from the experiments. At the end stage, tumor weight was registered and samples were collected for histological examination, metabolomics and/or FACS/sorter analysis.

RNA Extraction, cDNA Synthesis and qRT-PCR

To assess gene expression, RNA from cells was extracted with a RNeasy Minikit (Qiagen) according to the manufacturer's instructions and resuspended in 304 RNase-free water. RNA concentration was measured with the Nanodrop 2000 (Thermo Scientific). Reverse transcription of cDNA was performed with a QuantiTect Reverse Transcription Kit (Qiagen) or a SuperScript™ III First-Strand Synthesis System (Invitrogen), according to manufacturer's protocol. cDNA, primers mix and PowerUp™ SYBR Green Mix (Applied Biosystems) or TaqMan™ Fast Universal PCR Master Mix (Applied Biosystems) were prepared according to manufacturer's instructions. A total volume of 124 was pipetted into a 96-well MicroAmp plate (Applied Biosystems) and analysed using the QuantStudio™ 12K Flex Real-Time PCR System (Applied Biosystems). Gene transcription was presented as number gene mRNA copies relative to the housekeeping gene GADPH.

FACS Analysis

Tumor-bearing mice were sacrificed by cervical dislocation and perfused with saline to remove circulating immune cells. Tumors were harvested and minced in αMEM medium (Lonza) supplemented with 5% FBS, 1% Pen/Strep, 50 μM β-mercaptoethanol (Gibco), 5U/mL DNase I (Qiagen), 0.85 mg/mL Collagenase V (Collagenase from Clostridium histolyticum, Sigma-Aldrich), 1.25 mg/mL Collagenase D (Collagenase from Clostridium histolyticum, Roche) and 1 mg/mL Dispase II (Gibco) and incubated for 30 minutes at 37° C. The digested tissue was filtered using a 70 μm pore sized mesh strainer and cells were centrifuged 5 minutes at 300 xg. The samples were resuspended in Red Blood Cell Lysing Buffer Hybri-Max™ (Sigma-Aldrich) for 30 seconds, inactivated with FACS buffer (PBS containing 2% FBS and 2 mM EDTA) and centrifuged. The cell pellets were resuspended in FACS buffer and filtered with a 40 μm pore sized mesh strainer. Cells were resuspended in FACS buffer and for the intracellular measurement of interferon γ (IFN-γ) and granzyme B (GZMB), single-cell suspensions were culture in RPMI medium supplemented with 10% FBS, 1% glutamine, 1% Pen/Strep and stimulated with phorbol 12-myristate 13-acetate (PMA)/ionomycin Cell Stimulation Cocktail (eBioscience™, 1:500) in the presence of Brefeldin A (BioLegend®, 1:1000) or Monensin (eBioscience™, 1:1000) for 4 h at 37° C. Cells were then incubated for 15 minutes at 4° C. with Mouse BD Fc Block purified Rat Anti-Mouse CD16/CD32 mAb (BD Pharmingen™) and stained with the following antibodies for 30 minutes at 4° C.: Fixable viability dye (eFluor™ 506, eBioscience™), anti-CD45 (30-F11, APC-Cy7 or FITC, BioLegend®), anti-CD11b (M1/70, PerCP-Cy5.5, BioLegend®; eFluor™ 450, eBioscience™; or PE, BD Biosciences), anti-TCR-13 chain (H57-597, BV421, BD Biosciences), anti-CD4 (RM4-5, PE, BioLegend®), anti-CD8 (53-6.7, PE-Cy7, eBioscience™ or 53-6.7, APC-Cy7, BioLegend®), anti-CD69 (H1.2F3, APC, eBioscience™), anti-F4/80 (BM8, Alexa Fluor 488, eBioscience™; PerCP-Cy5.5, BioLegend®; or BV421, BioLegend®), anti-IFN-γ (XMG1.2, PE-Cy7, eBioscience™), anti-GZMB (GB11, Alexa Fluor 647, BioLegend®), anti-MHC-II (M5/114.15.2, APC, eBioscience™; or PerCP-eFluor 710, eBioscience™), anti-CD11c (N418, PE-Cy7, eBioscience™), anti-CD206 (MR5D3, Alexa Fluor 647, Bio-Rad), anti-Ly-6G (1A8, PE, BD Pharmingen™) anti-CD335 (NKp46) (29A1.4, APC, BioLegend®), anti-Foxp3 (FJK-16s, APC, eBioscience™) and anti-CD25 (PC61.5, PE-Cy7, eBioscience™). Cells were subsequently washed and resuspended in cold FACS buffer before FACS analysis or flow sorting by a FACS Verse or FACS Aria (BD Biosciences), respectively.

Alternatively, 100 μl of blood from tumor-bearing mice were resuspended in Red Blood Cell LysingBufferHybri-Max™ for 60 seconds, centrifuged and resuspended in FACS buffer. Cells were stained with the following cocktail of antibodies for 30 minutes at 4° C.: Zombie NIR™ Fixable Viability Dye (APC-Cy7, BioLegend®), anti-TCR-13 chain (H57-597, BV421, BD Biosciences), anti-CD4 (RM4-5, PerCP-Cy5.5, BioLegend®), anti-CD8 (53-6.7, PE-Cy7, eBioscience™), anti-CD44 (IM7, BV510, BioLegend®) and anti-CD62L (MEL-14, APC, eBioscience™). Cells were resuspended in Red Blood Cell Lysing Buffer Hybri-Max™, centrifuged and resuspended in FACS buffer before FACS analysis by a FACS Verse.

Fluorescence minus one (FMO) controls, unstained control and single staining controls were also performed in order to ensure proper gating of populations. Data were analysed by Flowjo (TreeStar).

Bone Marrow-Derived Macrophages (BMDMs) and Dendritic Cells (BMDCs)

Murine bone marrow-derived macrophages (BMDMs) were derived from bone marrow precursors as described before (Spranger et al. 2016, Proc Natl Acad Sci USA 113:E7759-E7768). Briefly, bone marrow cells (1×10⁷) were cultured in a volume of 5 ml in a 6-well plate in RPMI supplemented with 20% FBS, 30% L929 conditioned medium as source of M-CSF, 1% glutamine, 1 mM HEPES and 1% Pen/Strep. After 3 days of culture, additional 3 ml of differentiation medium were added.

Alternatively, at day 7, BMDMs were differentiated towards “M1”-like macrophages with 20 ng/ml IFN-γ and 100 ng/ml LPS (Sigma-Aldrich), or towards “M2”-like macrophages with 10 ng/ml IL-4 (eBioscience™).

At day 7/8, BMDMs were harvested with ice cold Ca²⁺- and Mg²⁺-free PBS. The cells obtained were uniformly macrophages as assessed by FACS, using the myeloid cell-specific marker CD11b and the pan-macrophage-specific marker F4/80.

For murine bone marrow-derived dendritic cells (BMDCs), 1×10⁷ bone marrow cells were cultured during 8 days without disturbing in a volume of 6 ml in a 10 cm Petri dish (non-tissue culture treated, bacterial grade) in RPMI supplemented with 10% FBS, 1% Glutamine, 25 mM HEPES, 1% NEAA, 1% Sodium Pyruvate, 1% Pen/Strep, 55 μM β-Mercaptoethanol and 100 ng/ml recombinant human Flt3L-Fc (BioXcell). At day 8, BMDCs were harvested with ice cold Ca²⁺- and Mg²⁺-free PBS. The cells obtained were uniformly dendritic cells as assessed by FACS, using the myeloid cell-specific marker CD11b and the specific markers CD11c and MHC-II.

Alternatively, macrophages were derived from BM precursors as described before (Meerpohl et al., 1976). Briefly, BMDMs (1.6×10⁶ cells/ml) were cultured in a volume of 6 ml in a 10 cm Petri dish (non tissue culture treated, bacterial grade) in DMEM supplemented with 20% FBS and 30% L929 conditioned medium as a source of M-CSF. After 3 days of culture, an additional 3 ml of differentiation medium was added. At day 7, macrophages were detached with ice cold PBS and used for electroporation and migration assay subsequently.

Cancer Cells Conditioned Media

After 48 hours, the conditioned media of NT and CDA KD Panc02 cells cultured in DMEM medium supplemented with 10% FBS and 1% Pen/Strep was collected.

BMDM Migration Assay

In the macrophage migration assays, 1×10⁵ murine BMDMs were seeded on 8 μM polycarbonate membranes (Transwell; Costar) with or without 10 μM MRS2578. The bottom chambers contained DMEM with or without 100 μM UDP, 100 μM Uridine (Sigma-Aldrich) or 100 μM Cytidine (Sigma-Aldrich), or alternatively, conditioned media from NT or CDA KD cells. When indicated, 2×10⁵ NT and CDA KD cells were seeded in the bottom chambers 36 h prior to the macrophage migration in DMEM supplemented with 2% FBS and 1% P/S, and upon that incubation period, 100 μM UDP were added to the chamber.

After 6 h incubation, the cells were removed from the top of each membrane with a cotton stick and migrated cells were fixed in 4% PFA, washed in PBS, stained with crystal violet (2.5 g/L) and Hoechst, and mounted on glass slides with ProLong Gold mounting medium without DAPI. Images were acquired with Olympus BX41 microscope and CellSense imaging software.

BMDM electroporation. Silencing of P2ry6 or P2ry14 was achieved by electroporation with specific siRNAs. Briefly, 8×10⁶ BMDMs were resuspended in 500 μl of Opti-MEM and were electroporated (250V, 950 mF, 0) with 100 μmol of each of three siRNAs in combination. After electroporation, medium was replaced with DMEM supplemented with 10% FBS, 1% Pen/Strep and 2 mM Glutamine (Gibco). Upon 24 h of incubation at 37° C. in a 5% CO₂ humidified atmosphere, migration assay was performed. In the macrophage migration assays, 2×10⁵ murine electroporated BMDMs were seeded on 8 μM polycarbonate membranes (Transwell; Costar). The bottom chambers contained DMEM supplemented with 2% FBS, with or without 100 μM UDP. After 6 h incubation, the cells were removed from the top of each membrane with a cotton stick and migrated cells were fixed in 4% PFA, washed in PBS, stained with crystal violet (2.5 g/L) and mounted on glass slides. Images were acquired with Olympus BX41 microscope and CellSense imaging software.

Commercially available siRNAs were purchased from ID Technology or Invitrogen (Scrambled control) and their assay IDs are listed below:

| siRNA | ID |
|---|---|
| P2y6r | mm.Ri.P2ry6.13.1, mm.Ri.P2ry6.13.2, mm.Ri.P2ry6.13.3 |
| P2y14r | mm.Ri.P2ry14.13.1, mm.Ri.P2ry14.13.2, mm.Ri.P2ry14.13.3 |
| Scrambled | Negative control REF 462002 |
| control | |

Statistical Analysis

Data entry and all analyses were performed in a blinded fashion. All statistical analyses were performed using GraphPad 7 Prism software on mean values, calculated from the averages of technical replicates. Statistical significance was calculated by two-tailed unpaired t-test on two experimental conditions or two-way ANOVA when repeated measures were compared, with p<0.05 considered statistically significant. Detection of mathematical outliers was performed using the Grubbs' test in GraphPad. Sample sizes for all experiments were chosen based on previous experience and material availability. Independent experiments were pooled and analysed together whenever possible as detailed in figure's legends. All graphs show mean values±standard error of the mean (SEM).

1. A method of treating or prohibiting the progression of cancer in a subject, the method comprising:
administering to the subject:
a specific inhibitor of the pyrimidinergic receptor P2Y6 (P2Y6) and;
an immune checkpoint inhibitor.

2. The method according to claim 1 wherein the cancer is not or only partially responsive to immune checkpoint inhibitor therapy.

3. A composition comprising:
an immune checkpoint inhibitor and;
a specific pyrimidinergic receptor P2Y6 inhibitor.

4. (canceled)

5. (canceled)

6. A composition comprising:
an isolated P2Y6 knock-out macrophage or;
an isolated macrophage conditionally expressing a specific P2Y6 inhibitor.

7. (canceled)

8. The composition of claim 6, wherein the composition further comprises an excipient.

9. The composition of claim 6, wherein the composition further comprises an immune checkpoint inhibitor.

10. (canceled)
